(19) United States
(12) Patent Application Publication
(10) Pub. No.: US 2026/0298868 A1
(43) Pub. Date: Oct. 1, 2026

(54) ELECTROPHORESIS CLAMP APPARATUS & SYSTEMS

(71) Applicant: Life Technologies Corporation, Carlsbad, CA (US)

(72) Inventors: Michael MORELAND, Redmond, WA (US); Jordan THOMPSON, Chino, CA (US); Marie MURAKAMI, Encinitas, CA (US); Ken CHAE, Encinitas, CA (US)

(21) Appl. No.: 19/562,983

(22) Filed: Mar. 11, 2026

Related U.S. Application Data
(60) Provisional application No. 63/781,536, filed on Apr. 1, 2025.

(51) Int. Cl. G01N 27/447; G01N 33/487

(52) U.S. Cl. CPC G01N 27/44704; G01N 33/48707

(57) ABSTRACT

The present disclosure provides systems and devices for gel electrophoresis including electrophoresis clamp assemblies also known as electrophoresis cores. Methods for electrophoresis using the systems and devices of the disclosure are also provided.

### PRIORITY

This application claims priority of U.S. Provisional Application No. U.S. 63/781,536 filed on Apr. 1, 2025, the entire content of which is incorporated herein by reference.

### FIELD

The present specification relates generally to apparatus, devices, systems, and methods for electrophoresis. More particularly, the specification relates to clamping apparatus for placing electrophoresis cassettes into electrophoresis tanks and methods of use thereof.

### BACKGROUND

Electrophoresis is a common procedure for the separation of biological molecules (biomolecules), such as nucleic acids, DNA, RNA, polypeptides and proteins based on their size and charge. In gel electrophoresis, biomolecules are separated into distinct bands by an electric field that causes the molecules to migrate through a filtering matrix that typically comprises a gel. Electrophoresis cassettes comprise a filtering matrix (such as a gel), that is sandwiched between two glass or plastic plates (typically forming a slab of filtering matrix between the two plates). Gels have an open molecular network structure defining pores that are saturated with an electrically conductive buffered solution including a salt. These pores are large enough to enable passage of biomolecules migrating through the gel in response to an electrical field. Several types of gels can be used for electrophoresis such as, but not limited to, polyacrylamide gels, agarose gels, starch gels and the like.

During electrophoresis, an electrophoresis gel, e.g., in a pre-cast or a self-cast electrophoresis cassette, is typically loaded with samples containing biomolecules and a tracking dye and placed in a chamber, typically known as an electrophoresis tank, the electrophoretic system having a cathode and an anode in contact with one or more buffer solutions that enable formation of an electric field across the gel when connected to an electrical power supply. The electric field so generated, is applied across the gel and consists of a negative charge at one end and a positive charge at the other end, causing sample biomolecules and tracking dye to separate from each other and migrate toward the bottom of the gel. Biomolecules of interest form distinct bands along the gel as they separate from each other. The electrophoresis is halted before the biomolecule of interest reaches the end of the gel or reached a position/location in the gel as desired by a user.

For maintaining a uniform electric field and efficient electrophoresis in an electrophoresis tank, a clamp apparatus/assembly (also known as a core or an electrophoresis core), that can hold in place one or more electrophoresis gel cassettes in an electrophoresis tank is used. While several clamps are described in the art, there is a need for better clamp apparatus designs that can be easier to manufacture and more efficient at electrophoresis.

### SUMMARY

In some embodiments, apparatus, systems, devices, and methods described herein address problems in the art by providing a versatile electrophoresis clamp assembly (also referred to herein as a “core” or “electrophoresis core” or “companion core”) that can be used with a variety of gel cassettes. Electrophoresis clamp assemblies of the present disclosure, additionally have features that improve electrophoresis run time, are more user friendly, and cheaper to manufacture.

In one embodiment, the present disclosure describes an electrophoresis clamp assembly comprising: a body, an electrode assembly comprising an anode and a cathode, and a pair of clamps, that can accommodate one or more electrophoresis cassettes. In some embodiments, an electrophoresis clamp assembly of the present disclosure can be used to place one or more electrophoresis cassettes comprising biomolecules for electrophoresis in an electrophoresis tank.

In some embodiments, the present disclosure describes an electrophoresis system that comprises at least one electrophoresis clamp assembly of the disclosure, an electrophoresis tank and at least one electrophoresis cassette (including both self-pour/self-cast cassettes and pre-cast cassettes). In some embodiments, the present disclosure describes an electrophoresis system that comprises at least two electrophoresis clamp assemblies of the disclosure, at least two electrophoresis cassettes, and an electrophoresis tank. The at least one electrophoresis clamp assembly of a system of the disclosure is configured such that a lid of an electrophoresis tank can fit in only one orientation when the clamp assemblies of the disclosure are placed into an electrophoresis tank to ensure correct polarity of the electrophoresis run direction when the system is in use.

In some embodiments, a body of an electrophoresis clamp assembly of the present disclosure, comprises a flat bottom part that is contiguous with two upright parts on the left and right sides, and a horizontal part that spans the center and is contiguous with portions of the two upright parts.

In some embodiments, the body of an electrophoresis clamp assembly of the present disclosure, comprises one or more base tabs configured to accommodate one or more electrophoresis gel cassettes. In some embodiments, an electrophoresis clamp assembly of the disclosure comprises at least two base tabs. In some embodiments, an electrophoresis clamp assembly of the disclosure comprises two base tabs and can accommodate two electrophoresis cassettes. In some embodiments, two electrophoresis cassettes are accommodated on the front and back side of an electrophoresis clamp assembly of the disclosure.

In some embodiments, an electrophoresis cassette can be a blank electrophoresis cassette, i.e., an electrophoresis cassette without any gel inside which is also known as a buffer dam. A blank electrophoresis cassette or a buffer dam enables a user to run either one electrophoresis gel or three electrophoresis gels in a system of the disclosure, as desired by a user. Alternatively, two or four electrophoresis cassettes with gels can be run in systems of the disclosure.

The one or more base tabs are further configured to provide support to an electrophoresis clamp assembly of the disclosure. For example, the one or more base tabs can function as “feet” and provide a stable base.

In some embodiments, a pair of clamps of an electrophoresis clamp assembly of the present disclosure, are adapted to reversibly engage with portions of the body. In embodiments, clamps reversibly engage with the outer sides of the right upright part and the left upright part of the body. The clamps are detachable from the body of an electrophoresis clamp assembly. Clamps of the disclosure are configured to enclose one or more electrophoresis cassettes when the clamps are engaged with portions of the body. In some embodiments, clamps of the disclosure are configured to clamp one, two, three or four electrophoresis cassettes, including combinations of electrophoresis cassettes with gels and biomolecules and buffer dams.

In some embodiments, the clamps are wing clamps. In some embodiments, the two bottom ends of the clamp portions of the electrophoresis clamp assembly of the disclosure are flared.

In some embodiments, the two clamps of the electrophoresis clamp assembly of the disclosure comprise a pair of grips on the top.

In some embodiments, the cathode of an electrophoresis clamp assembly of the disclosure comprises a bar shaped metal. The cathode metal can be stainless steel, gold, silver, nickel alloys, platinum or other metals that are good conductors, inert, and resistant to electrolytic type of corrosion. In some embodiments, a cathode of an electrophoresis clamp assembly spans across the horizontal center part of the body. In some embodiments, the cathode further comprises a protrusion on one side. The placement of the cathode protrusion ensures that a lid of an electrophoresis gel tank into which an electrophoresis clamp assembly is placed can fit in only one orientation.

In some embodiments, a cathode of the electrophoresis clamp assembly is connected to at least one of the upright parts of the body. In some embodiments, the cathode can be held in place by a hex-nut or similar screw. In some embodiments, the cathode can be connected to a power supply by a banana plug or similar connector.

In some embodiments, an anode of an electrophoresis clamp assembly of the disclosure comprises a bare wire made of platinum or other metals.

In some embodiments of an electrophoresis clamp assembly of the disclosure, when clamps are locked to enclose an electrophoresis cassette, at least one chamber is formed between the walls of the one or more electrophoresis cassettes and one or more body portions of the clamp assembly. This chamber is referred to herein as a cathode buffer chamber and during use a cathode buffer chamber can enclose an inside core volume of an electrophoresis buffer. The remainder of the electrophoresis buffer in the electrophoresis tank is referred to herein as an anode buffer chamber which encloses an outside core volume of electrophoresis buffer.

In some embodiments, an electrophoresis clamp assembly of the disclosure further comprises one or more groves to accommodate one or more gaskets. Sealing of an electrophoresis cassette to an electrophoresis clamp assembly of the disclosure, is at least in-part facilitated by a gasket placed into a grove located on the body of the electrophoresis clamp assembly. In some embodiments, a groove has a U-like shape and has uniform dimensions all around. The groove accommodates a gasket which is in a similar U-like shape and tapers from a narrow bottom to a wider width towards the two top sides. Advantageously, the tapered gasket sealing surface aligns compression force of the clamps onto the electrophoresis cassettes at the center of the clamping assembly.

In some embodiments, the disclosure provides electrophoresis method comprising: clamping at least two electrophoresis cassettes to an electrophoresis clamp assembly described herein, placing the clamped electrophoresis clamp assembly with the electrophoresis cassettes into an electrophoresis tank, filling the electrophoresis tank with at least one electrophoresis buffer, loading at least one of the two electrophoresis cassettes with a sample comprising a biomolecule and optionally a loading dye, placing an electrophoresis tank lid onto the electrophoresis tank in such a way that the anode and cathode are connected in the right polarity to an external power supply via metal connectors in the tank lid, switching on the power supply and running the electrophoresis till the sample biomolecules in the one or more electrophoresis cassettes have been electrophoresed.

In some embodiments of the method, one of the two electrophoresis cassettes is a buffer dam. In some embodiments of the method, the electrophoresis gel clamp assembly is an electrophoresis core. In some embodiments of the method, two electrophoresis clamp assemblies are used. In some embodiments of the method, one electrophoresis clamp assembly is an electrophoresis core and the second electrophoresis clamp assembly is a companion core.

In some embodiments of the method, up to two electrophoresis cassettes can be run. In some embodiments of the method, up to four electrophoresis cassettes can be run. In some embodiments of the method, one of the two or one of the four electrophoresis cassettes is a buffer dam.

One or more advantages of the electrophoresis clamp assembly and electrophoresis system comprising an electrophoresis clamp assembly of the disclosure include one or more of the foregoing. In some embodiments, systems and assemblies of the disclosure enable users to run up to four mini-gel cassettes in a single tank or run two-mini-gel cassettes in a single tank. In some embodiments, the stainless steel metal cathode bar which spans the clamp assembly on the top side provides an improvement in electrophoresis run time and also lowers the manufacturing cost of the electrophoresis clamp assembly as compared to currently available electrophoresis clamp assemblies. Anode of a device of the disclosure is typically made of platinum wire (or other metal) and spans across the bottom of the clamp assembly. The combination of electrodes results in efficient electrophoresis. In embodiments, the cathode bar has a protrusion which prevents placing a lid on the electrophoresis tank in an orientation that could inadvertently reverse the polarity of the electric field needed electrophoretic separation of biomolecules. Accordingly, the present electrophoresis clamp assembly and systems comprising the same prevents inadvertent reverse placing of a lid of the electrophoresis tank and thereby prevents reversing orientation of the electric field. In embodiments, use of an anode comprising a bare platinum (or other metal) wire that is not enclosed in a Teflon or other plastic sheath, provides improvements to electrophoresis performance. In some embodiments, clamp assemblies of the disclosure have a raised “+” and “−” feature on top of the body which provides an additional electric field orientation cue for users, including users who may be color blind. In some embodiments, clamp assemblies of the disclosure can have red or black colored markings on the top of the body, where black identifies a cathode and red identifies an anode, to provide an additional electrical field orientation cue for users.

These and other features of the present teachings will become more apparent from the detailed description in sections below.

### BRIEF DESCRIPTION OF THE DRAWINGS

One or more embodiments of the present disclosure may be better understood in reference to one or more the drawings below. The skilled artisan will understand that the drawings, described below, are for illustration purposes only. The drawings are not intended to limit the scope of the present teachings in any way.

FIG. 1A is a schematic representation of a perspective view of an exemplary electrophoresis clamp assembly, in an un-locked position, according to one embodiment;

FIG. 1B is a schematic representation of a perspective view of another exemplary electrophoresis clamp assembly, in an un-locked position, according to one embodiment;

FIG. 1C depicts a body of an electrophoresis clamp assembly, according to one embodiment;

FIG. 1D depicts a gasket with the insert showing tapering of gasket, according to one embodiment;

FIG. 2 is a bottom perspective view of an electrophoresis clamp assembly in an un-locked position, such as that shown in FIGS. 1A and 1B, according to one embodiment;

FIGS. 3A and 3B, depict exemplary clamps of an electrophoresis clamp assembly of the disclosure, according to one embodiment;

FIG. 4A is a front perspective view of an electrophoresis clamp assembly in a locked position, according to one embodiment;

FIG. 4B is a front perspective view of an electrophoresis clamp assembly in a locked position, according to one embodiment;

FIG. 5 is a bottom perspective view of electrophoresis clamp assemblies in a locked position, such as those shown in FIGS. 4A and 4B, according to one embodiment;

FIGS. 6A and 6B are front perspective views of exemplary embodiments of electrophoresis clamp assemblies of the disclosure, in an un-locked position, with two electrophoresis cassettes;

FIG. 7A is a bottom perspective view of an electrophoresis clamp assembly in an un-locked position, with two electrophoresis cassettes, such as that shown in FIGS. 6A and 6B;

FIG. 7B is a front view of an electrophoresis clamp assembly in an un-locked position, such as that shown in FIG. 7A, and FIG. 7C shows section AA of FIG. 7B detailing engagement of electrophoresis cassettes with base tabs and a backstop boss, according to one embodiment;

FIGS. 8A and 8B are front perspective views of exemplary embodiments of electrophoresis clamp assemblies of the disclosure, in a locked position, enclosing two electrophoresis cassettes;

FIG. 9 depicts a bottom perspective view the electrophoresis clamp assemblies of FIG. 8A and FIG. 8B, showing two electrophoresis cassettes;

FIG. 10 depicts a perspective view of an exemplary electrophoresis system, showing two exemplary electrophoresis clamp assemblies with two electrophoresis cassettes each, placed inside a chamber of an electrophoresis tank, to form a system of the disclosure wherein both clamps are in a locked position, according to one embodiment;

FIG. 11A and FIG. 11B, depict successful electrophoresis in 4-12% NuPAGE Bis-Tris gels in an electrophoresis core of the present disclosure, while FIG. 11C and FIG. 11D show comparative results of electrophoresis of identical samples in 4-12% NuPAGE Bis-Tris gels when the electrophoresis was run in a Mini-Gel Tank;

FIG. 12A and FIG. 12B, depict successful electrophoresis in 4-12% Bolt Bis-Tris Plus gels in an electrophoresis core of the present disclosure, while FIG. 12C and FIG. 12D show comparative results of electrophoresis of identical samples in 4-12% Bolt Bis-Tris gels when the electrophoresis was run in a Mini-Gel Tank;

FIG. 13A and FIG. 13B, depict successful electrophoresis in 4-20% Novex Tris-Glycine Plus gels in an electrophoresis core of the present disclosure, while FIG. 13C and FIG. 13D show comparative results of electrophoresis of identical samples in 4-20% Novex Tris-Glycine Plus gels when the electrophoresis was run in a Mini-Gel Tank;

FIG. 14A and FIG. 14B, depict successful electrophoresis in 3-8% NuPAGE Tris-Acetate gels in an electrophoresis core of the present disclosure, while FIG. 14C and FIG. 14D show comparative results of electrophoresis of identical samples in 3-8% NuPAGE Tris-Acetate gels when the electrophoresis was run in a Mini-Gel Tank; and

FIG. 15A and FIG. 15B, depict successful electrophoresis in 10-20% Novex Tricine gels in an electrophoresis core of the present disclosure, while FIG. 15C shows comparative results of electrophoresis of identical samples in 10-20% Novex Tricine gels when the electrophoresis was run in a Mini-Gel Tank.

### DETAILED DESCRIPTION OF THE DISCLOSURE

It is to be understood that both the foregoing general description and the following detailed description are exemplary and explanatory only and are not intended to limit the scope of the current teachings. In this application, the use of the singular includes the plural unless specifically stated otherwise. For example, the singular forms “a”, “an” and “the” as used in the specification also include plural aspects unless the context dictates otherwise. Similarly, any singular term used in the specification also mean plural or vice versa unless the context dictates otherwise.

Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range therebetween unless otherwise specifically stated. For example, “from 0.2 to 0.5” means 0.2, 0.3, 0.4, 0.5; ranges therebetween such as 0.2-0.3, 0.3-0.4, 0.2-0.4; increments there between such as 0.25, 0.35, 0.225, 0.335, 0.49; increment ranges there between such as 0.26-0.39; and the like.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Described herein are systems, devices, and methods for electrophoresis of biomolecules. Systems, devices and methods of the present disclosure overcome several of the problems in the art. In some embodiments, electrophoresis clamp systems and devices that can be used universally for gel electrophoresis in a variety of electrophoresis gel tanks is described.

### Devices and Systems

The present disclosure describes an electrophoresis clamp assembly device comprising: a body; an electrode assembly comprising an anode and a cathode; and a pair of clamps, that can accommodate one or more electrophoresis cassettes in an electrophoresis tank.

Embodiments of the present disclosure can be understood in reference to the exemplary drawings described in detail below. However, the drawings and features therein are not intended to limit the scope of the present teachings in any way and are only for illustrative purposes of understanding the various embodiments.

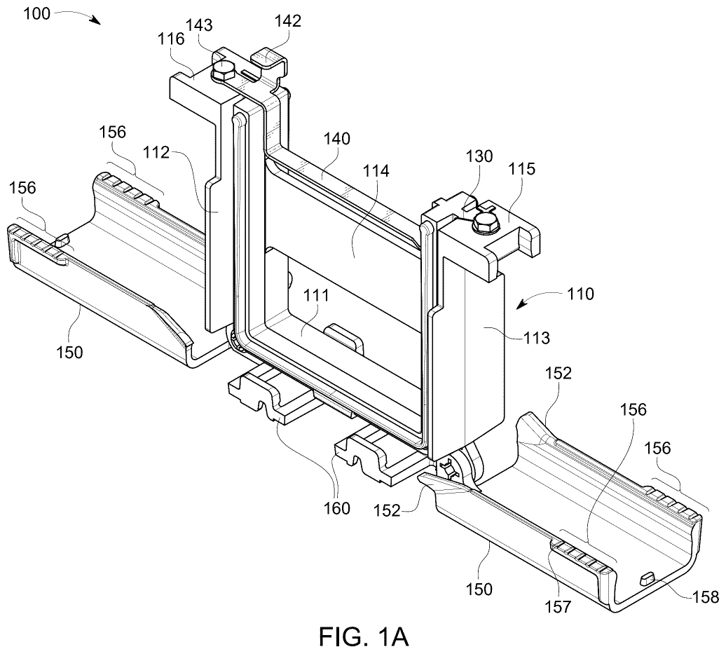

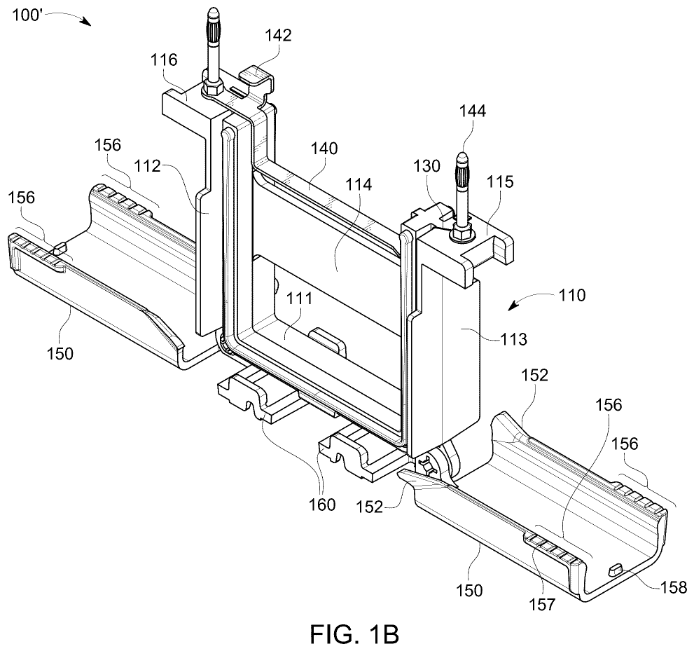

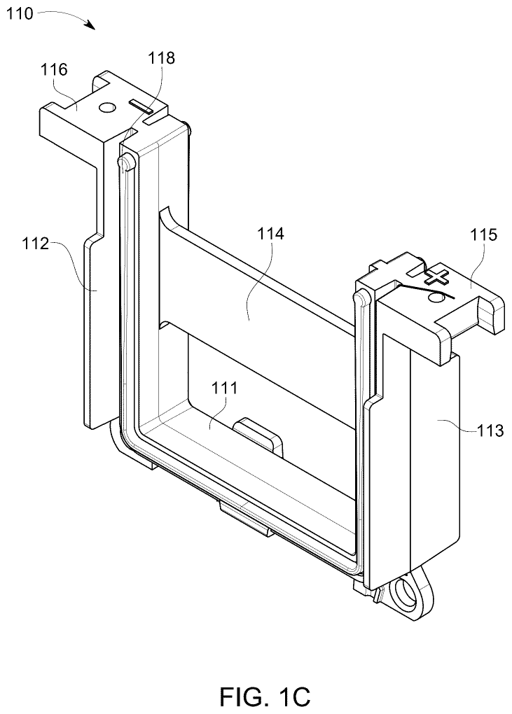

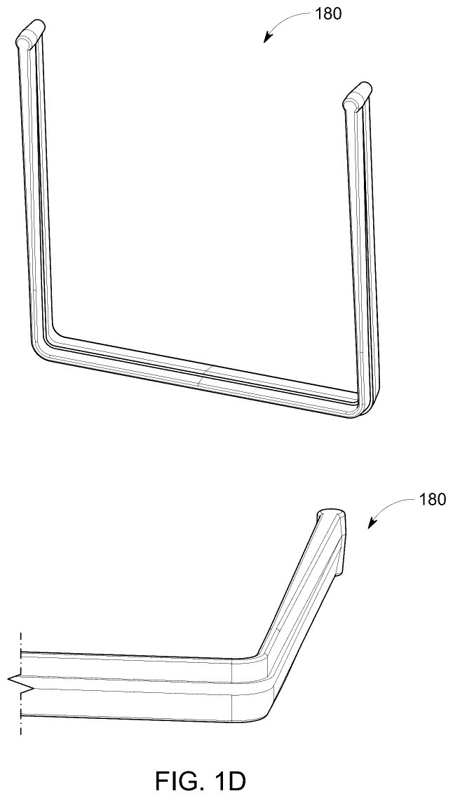

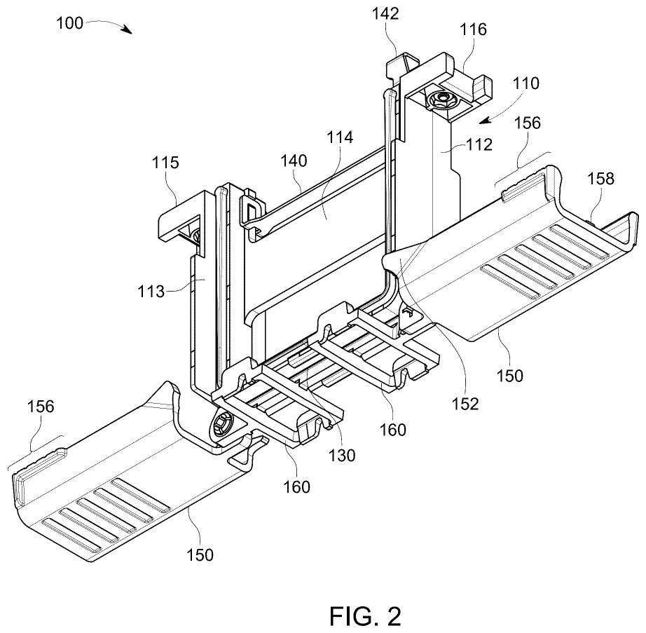

FIG. 1A is a schematic representation of a perspective view of an exemplary electrophoresis clamp assembly 100, comprising a body 110, a pair of clamps 150 and an electrode assembly (described further below), where clamps 150 are open or un-locked. Body 110 comprises a flat bottom part 111, that is contiguous with two upright parts that rise vertically on the left and right, left upright part 112 and right upright part 113 respectively, and a horizontal part 114 that spans the center of the body 110 and is contiguous with portions of the left and right upright parts. The electrode assembly comprises an anode 130 and a cathode 140. Cathode 140 comprises a bar shaped metal that spans the body 110. Cathode 140 is preferably comprised of a stainless-steel bar and the anode 130 is preferably comprised of platinum. In some embodiments, anode 130 is made of a bare platinum wire (also see FIGS. 2, 5, 9). However, other metals that are good conductors, inert, and resistant to electrolytic type corrosion, can be used to made anodes or cathodes and some examples include gold, silver, nickel alloys or platinum. Coated or plated aluminum, copper, or brass can be used to make anode 130 and cathode 140 where the coating/plating is to protect the electrodes from corrosion.

In some embodiments, cathode 140 further comprises a protrusion 142 on one side. Hex-screws 143 or any similar screws are used to attach cathode 140 to left extension 116 of left upright part 112. Similar screws are used to attach anode 130 to the right extension 115.

Electrophoresis clamp assembly 100 further comprises one or more base tabs 160 configured accommodate one or more electrophoresis cassettes (not depicted in this figure). Base tabs 160 are also configured to provide a stable base for electrophoresis clamp assembly 100. An electrophoresis clamp assembly 100 is also alternatively referred to as a “core” or a “companion core.”

FIG. 1B is a schematic representation of a perspective view of another embodiment of an exemplary electrophoresis clamp assembly 100′, with clamp 150 in an un-locked, or open or horizontal position. Electrophoresis clamp assembly 100′ has like parts and descriptions as in FIG. 1A, except that it has a banana plug connector 144 that enables an external electrical connection for cathode 140 and anode 130. This external electrical connection is mediated via an electrophoresis tank lid (not depicted) to a power source (not depicted). However, alternative electrical connections to a power source are possible via connector 144. An electrophoresis clamp assembly 100′ is alternatively referred to herein as an “electrophoresis core.”

FIG. 1C is a view of body 110 of an electrophoresis clamp assembly of the disclosure, with similar parts as described above. Body 110 of electrophoresis clamp assemblies of the disclosure further comprises one or more groves to accommodate one or more gaskets. For example, grove 118 on body 110 accommodates gasket 180. Groove 118 in some embodiments has a U-like shape. Groove 118 has the same width throughout. Sealing of an electrophoresis cassette 200 to an electrophoresis clamp assembly (100 or 100′), is at least in-part facilitated by gasket 180 placed into grove 118. Gasket 180, shown in FIG. 1D, has a U-like shape where the bottom portions are narrow. The two upright edges of gasket 180 are tapered such that the gasket increases in width toward the upright top end parts. Tapering of the gasket width is depicted in the portion of the gasket depicted in the insert in FIG. 1D. Advantageously, the sealing surfaces of tapered ends of gasket 180 align the compression force of clamps 150 onto electrophoresis cassettes 200 at the center of the clamping assemblies 100 or 100′.

FIG. 2 is a bottom perspective view of electrophoresis clamp assemblies, such as 100 or 100′, in an un-locked, or open or horizontal position, and has similar parts and descriptions as above.

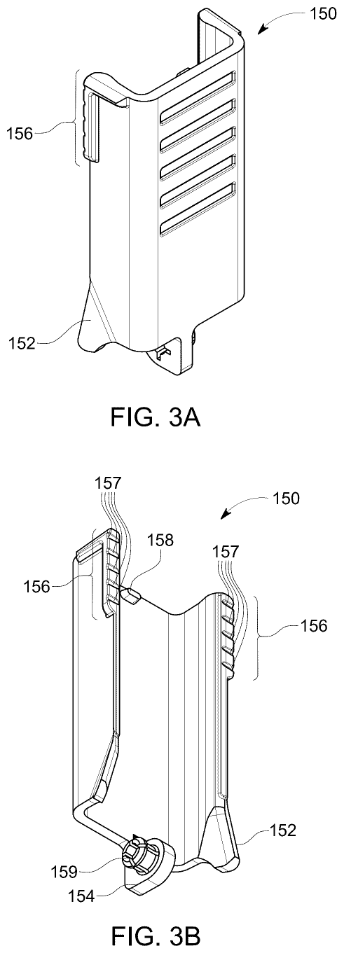

FIGS. 3A and 3B, depict exemplary clamps 150 of an electrophoresis clamp assemblies 100 or 100′ of the disclosure, according to one embodiment. Clamps 150, each have a flared bottom end 152. Top end of the clamps 150 have a pair of grips 156 on the top. Grips 156 have one or more ridges 157 that enable a user to grip the clamp while locking and unlocking clamps 150 to portions of body 110. Raised boss 158 (also called standoff feature 158) on the top inside part of clamps 150, maintains a small space between the clamp and the body that ensures clamp 150 is stopped in a vertical position when locked.

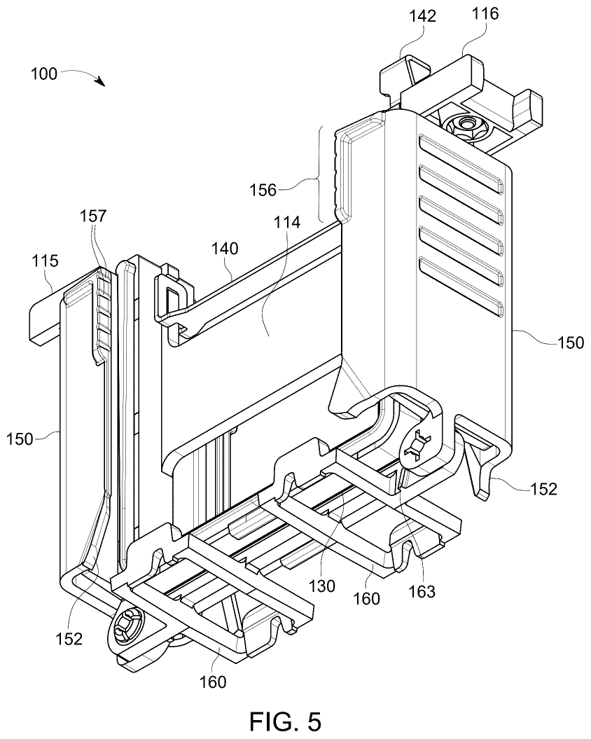

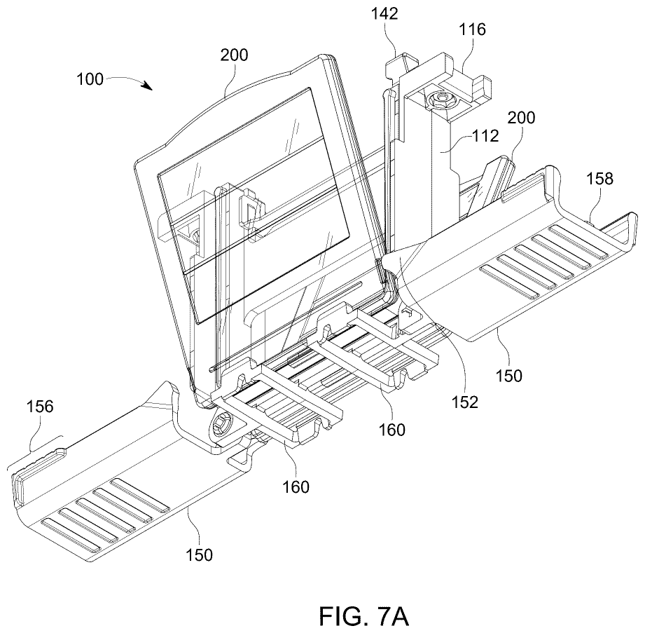

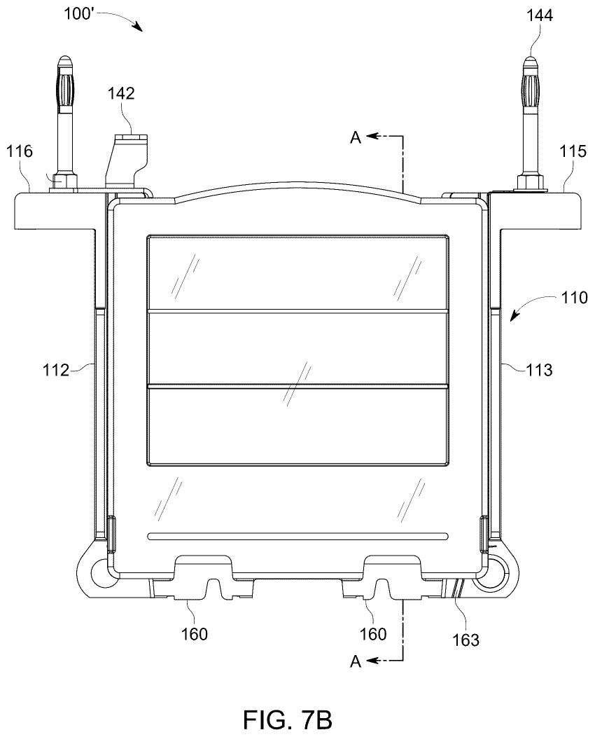

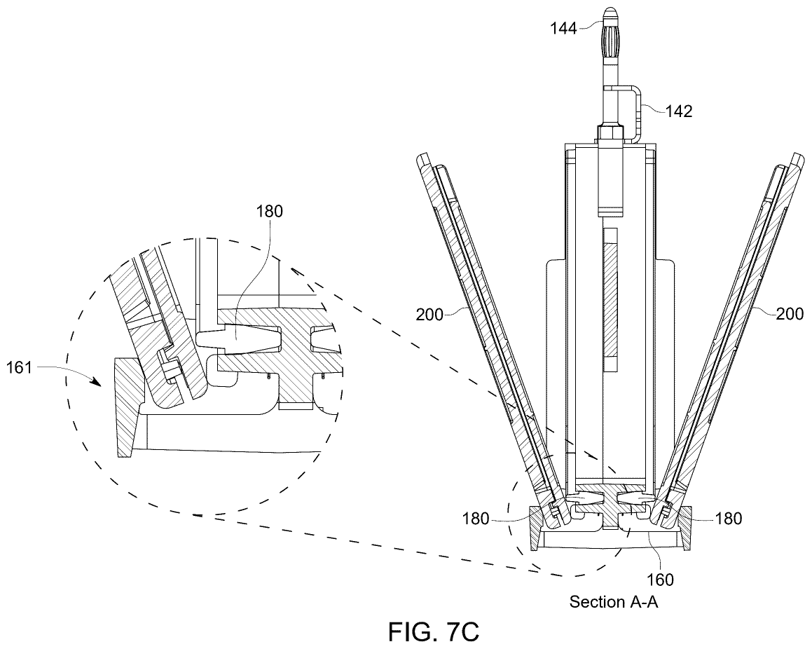

Clamp 150 can reversibly attach/clamp to the body part 110 via connector 159 also referred to as a pivot boss. Connector 159 facilitates a rotation motion of clamps 150 from the locked or vertical position to an unlocked/open or horizontal position of clamps 150. As clamp 150 rotates past the clamp open position (which corresponds to a horizontal position when the clamp apparatus is placed on a table top) the clamp will engage the raised stop rib 163 (see FIG. 5 and FIG. 7B)). Forcing clamp 150 against the raised stop rib 163 will cause the clamp 150 to detach from the clamp assembly. The function of the raised stop rib 163 and the pivot boss 159 are to protect the anode platinum wire 130 that extends at the bottom of body 110 from being crushed or broken from being pinched between the pivot flange 154 and the corresponding portion of body 110 that pivot boss 159 engages with. One advantage of a clamp assembly of the disclosure, in that neither the clamps 150 nor the body 110 of a clamp assembly 100 or 100′ will break and nor will the anode wire be broken or crushed. A user can simply reinsert clamp 150 via pivot boss 159 into body 110 of a clamp assembly of the disclosure if clamp 150 is inadvertently rotated past the raised boss 158 by the user.

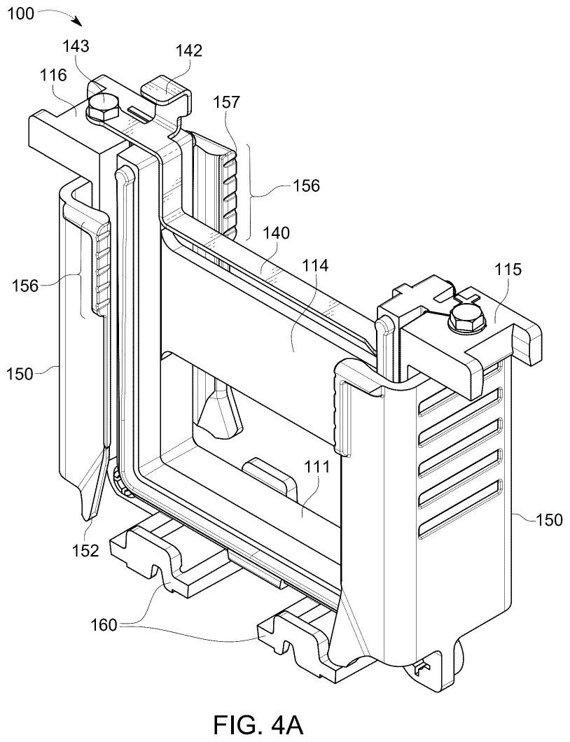

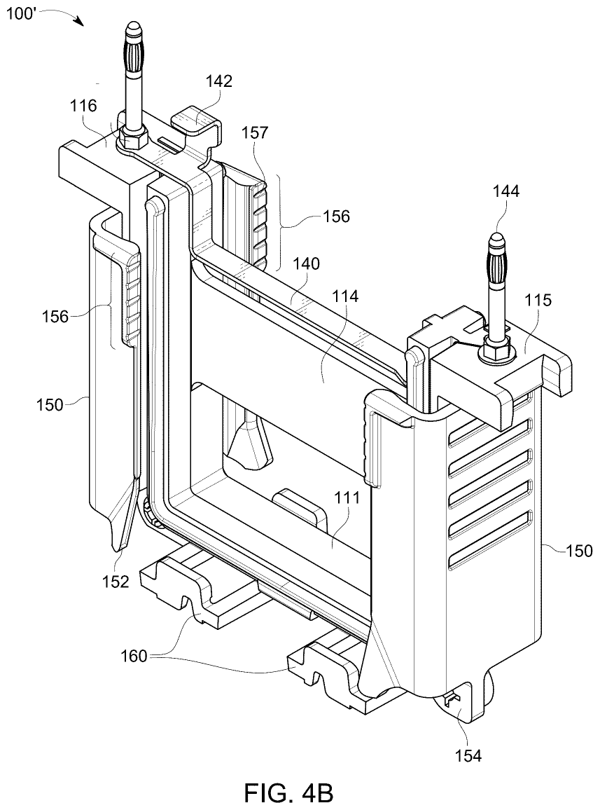

FIG. 4A is a front perspective view of an electrophoresis clamp assembly 100, with clamps 150 in a closed or locked position or vertical position. FIG. 4B is a front perspective view of an electrophoresis clamp assembly 100′, with clamps 150 in a closed or locked or vertical position.

FIG. 5 is a bottom perspective view of an electrophoresis clamp assembly such as 100 or 100′ with clamps 150 in a closed or locked or vertical position.

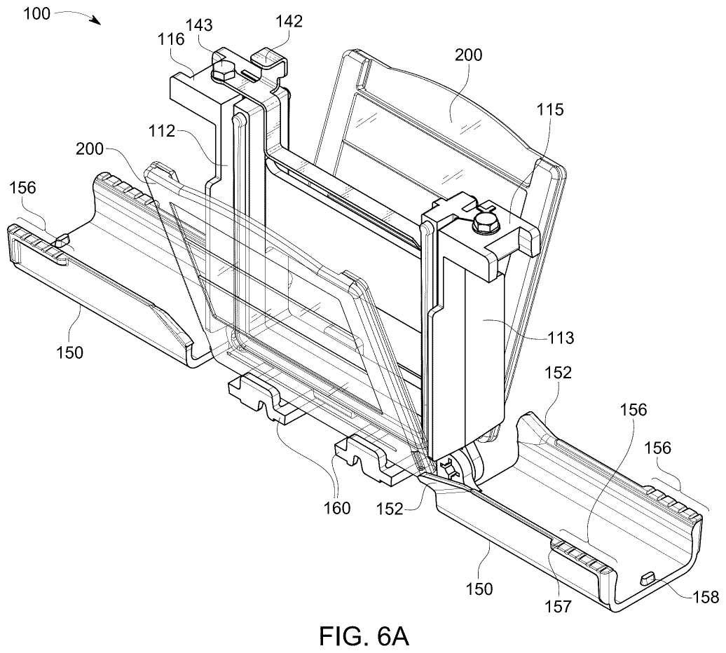

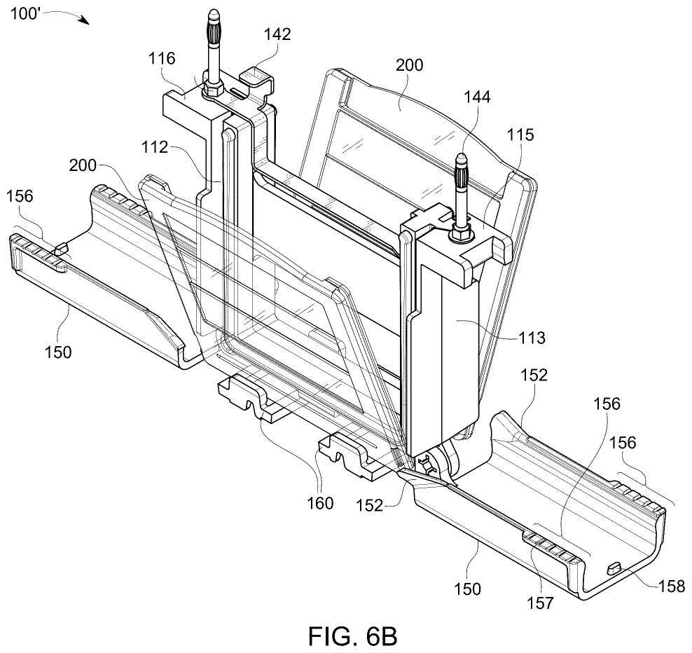

FIGS. 6A and 6B are front perspective views of exemplary embodiments of electrophoresis clamp assemblies 100 and 100′, wherein clamp 150 is in an open or un-locked position, with two electrophoresis cassettes 200 placed onto base tabs 160.

FIG. 7A is a bottom perspective view of an electrophoresis clamp assembly, such as 100 or 100′ wherein clamp 150 is in an open or un-locked position.

FIG. 7B is a view of an electrophoresis clamp assembly 100′ marking section AA. Sectional view of AA is depicted in FIG. 7C, showing details of engagement of electrophoresis cassettes 200 with base tabs 160, base tab feet 161 and gasket 180.

Gasket 180 can be a silicone rubber gasket. As described above, the gasket sealing surface is tapered in that it is narrow at the bottom side of the electrophoresis clamp body 110 and becomes wider towards the top on both upright sides of the body 112 and 113. The bottom portion of gasket 180 is of a uniform narrow width and then along each vertical upright left and right portions 112 and 113 the sealing surface of the gasket widens towards the top. Gasket 180 provides a unique tapering sealing surface that provides superior sealing engagement with electrophoresis cassettes 200.

Base tabs 160 can be seen in more detail in FIG. 7B. In some embodiments, base tabs 160 allow electrophoresis cassettes 200 to sit as low as possible in an electrophoresis tank, with arms that wrap around the tank's locating ribs (not shown). The width of base tabs 160 is designed to support electrophoresis cassettes 200 to minimize warping. This feature is not present in other electrophoresis clamp assemblies, such as Bio-Rad's Mini-PROTEAN Tetra electrode assemblies. The angled surfaces on the upper portion of base tabs 160 provide support and hold electrophoresis cassettes 200 before closing or locking the wing clamps 150. This mechanism works by drawing the electrophoresis cassettes 200 inward, pressing them into gaskets 180. Protrusions on the bottom angled surface of the lower portions of base tabs 160 guide the electrophoresis clamp with electrophoresis cassettes into the electrophoresis tank with locating ribs. The arms and notch features in the base tabs engage with these locating ribs to position and secure the cores within the tank. Properly lowering an electrophoresis cassette into the tank ensures optimal heat dissipation during an electrophoresis run, leading to optimal results. Additionally, protrusions 161 extend below the centerline of base tabs 160, functioning as feet to provide stability to the electrophoresis clamp assembly when placed on a bench top.

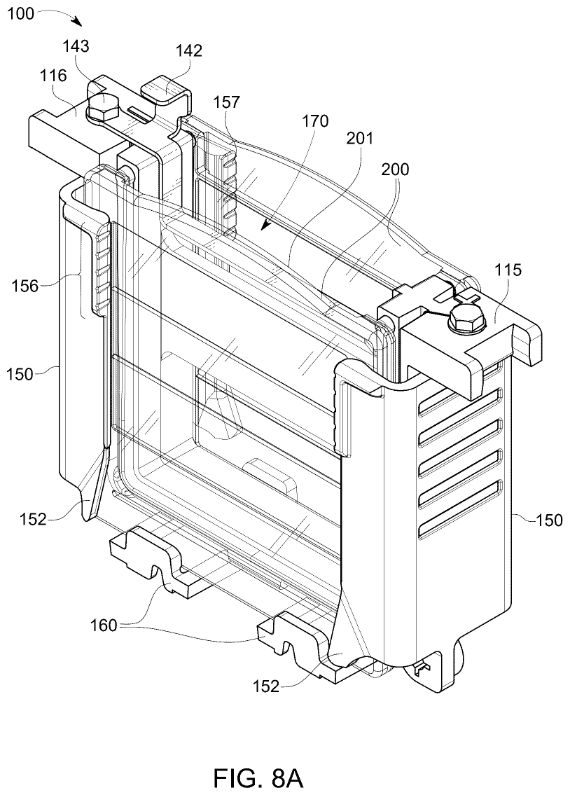

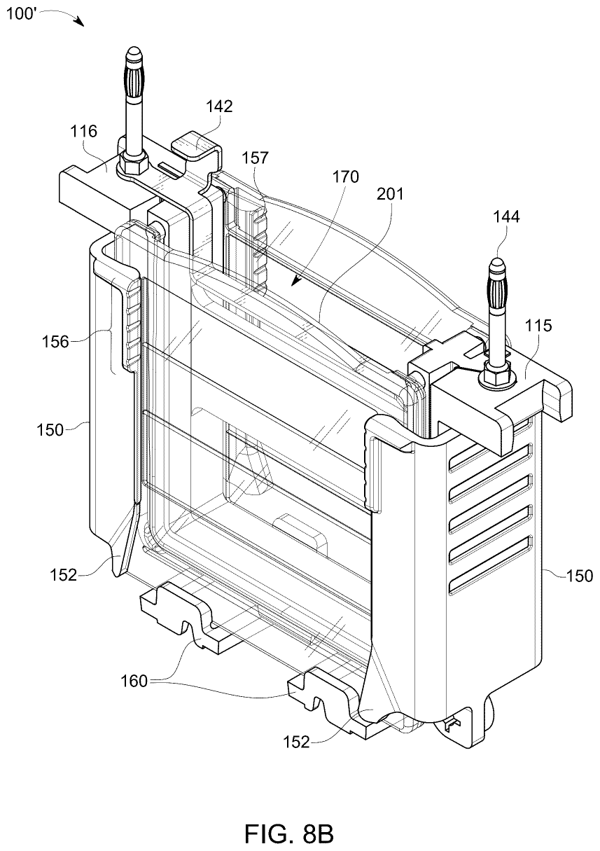

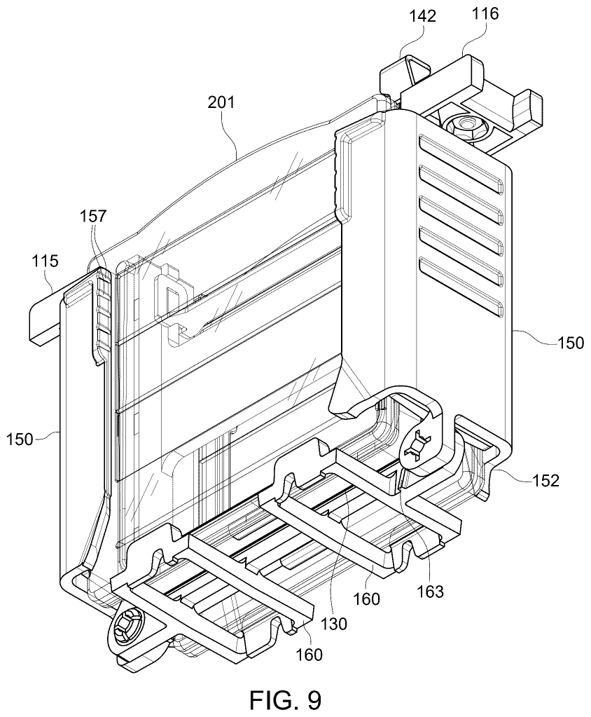

FIGS. 8A and 8B are front perspective views of exemplary embodiments of an electrophoresis clamp assemblies 100 and 100′, wherein clamp 150 is in a closed or locked position, enclosing two electrophoresis cassettes 200. FIG. 9 depicts a bottom perspective view of electrophoresis clamp assemblies 100 and 100′ of FIG. 8A and FIG. 8B, showing two electrophoresis cassettes 200 clamped in.

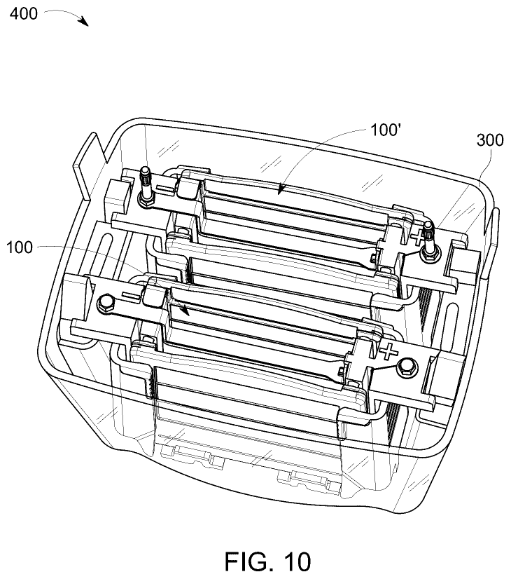

FIG. 10 depicts a perspective view of an exemplary electrophoresis system 400, showing two exemplary electrophoresis clamp assemblies 100 (also known as a “companion core”) and 100′ (also known as an “electrophoresis core”), with two electrophoresis cassettes 200 each, with clamps 150 in closed or locked positions, placed inside a chamber of an electrophoresis tank 300, to form a system of the disclosure.

In some embodiments of an electrophoresis system 400 of the disclosure, when clamps 150 are locked to enclose electrophoresis cassette 200, at least one chamber 170 is formed between the inner sides of the one or more electrophoresis cassettes 200 and one or more body portions of the clamp assembly 100 or 100′ (see FIG. 8B). Chamber 170 is referred to as a cathode buffer chamber and during use the cathode buffer chamber can enclose an inside core volume of an electrophoresis buffer. The remainder of the electrophoresis buffer in tank 300 is referred to as an anode buffer chamber. Up to four electrophoresis cassettes 200 can be run in system 400. In some embodiments in system 400, only three electrophoresis cassettes are placed and run and the fourth electrophoresis gel cassette that is placed into either the companion core 100 or the electrophoresis core 100′ is a buffer dam or a blank electrophoresis cassette.

In an alternative embodiment, not expressly depicted here, another exemplary electrophoresis system of the present disclosure can have one electrophoresis clamp assembly, such as 100′ which is also known as an electrophoresis core, enclosing two electrophoresis cassettes 200, with clamps 150 in closed or locked positions, placed inside a chamber of an electrophoresis tank 300, to form a system of the disclosure. In this embodiment, the companion core 100 is not present and the system can run up to two electrophoresis cassettes 200. In some embodiments of this system, only one electrophoresis cassette 200 is placed and run and the second electrophoresis cassette 200 that is placed into the companion core 100 is a buffer dam or a blank electrophoresis cassette.

In systems of the disclosure, electrophoresis clamp assemblies 100 and/or 100′ are configured such that a lid (not depicted) of an electrophoresis tank 300 can fit in only one orientation when the clamp assembly of the disclosure is placed into the gel tank to ensure correct polarity of the electrophoresis run direction when the system is in use.

### Methods of Use

In use an electrophoresis system of the present disclosure, comprising an electrophoresis clamp assembly to which are clamped electrophoresis cassettes, is placed into an electrophoresis tank filled with one or more electrophoresis buffers. Electrophoresis tanks have lids with metal connectors that connect to external power sources. As noted in sections above, electrophoresis clamp assemblies of the present disclosure are configured such that a lid of an electrophoresis tank can fit in only one orientation when the clamp assembly of the disclosure is placed into the tank to ensure correct polarity of the electrophoresis run direction when the system is in use. Accordingly, electrophoresis clamp assemblies and systems of the disclosure ensure electrophoretic run in the correct orientation. Electrophoresis clamp assemblies and systems of the disclosure are used to run electrophoresis of biomolecules in a sample till the sample biomolecules have been resolved based on their molecular weight, charge and/or pKₐ and combinations thereof.

In one embodiment, a method of the disclosure comprises, clamping at least two electrophoresis cassettes 200 to an electrophoresis clamp assembly of the disclosure, such as the electrophoresis core assembly 100′, placing the clamped or locked electrophoresis clamp assembly 100′ with the electrophoresis cassettes into an electrophoresis tank, filling the tank with at least one electrophoresis buffer, loading at least one of the two electrophoresis cassettes with a sample comprising a biomolecule and optionally a loading dye (the second electrophoresis cassette may either also comprise samples to be electrophoresed or can comprise a blank cassette also referred to herein as a buffer dam with no sample), placing an electrophoresis tank lid onto the tank in such a way that the anode and cathode electrodes are connected in the right polarity to an external power supply via metal connectors in the tank lid, switching on the power supply and running the electrophoresis till the sample biomolecules have been resolved based on their molecular weight. In one embodiment of this method, up to two electrophoresis gels can be run. In some embodiments, one electrophoresis gel and one buffer dam can be run.

In another embodiment, a method of the disclosure comprises, obtaining two electrophoresis clamp assemblies including an electrophoresis core 100′ and a companion core 100, clamping at least two electrophoresis cassettes 200 to each of the electrophoresis core and the companion core, placing the clamped or locked electrophoresis clamp assemblies (i.e., the companion core 100 and the electrophoresis core 100′ with the two cassettes 200 clamped in) into an electrophoresis tank 300, filling the tank 300 with at least one electrophoresis buffer, loading at least one of the four electrophoresis cassettes 200 with a sample comprising a biomolecule and a loading dye (the other electrophoresis cassette 200 may either also comprise samples to be electrophoresed or may be a buffer dam with no sample and/or gel), placing a electrophoresis tank lid (not depicted) onto the tank 300 in such a way that the anode and cathodes of the companion core and the electrophoresis core are connected in the right polarity to an external power supply via metal connectors in the tank lid, switching on the power supply and running the electrophoresis. In one embodiment of this method, up to four electrophoresis cassettes 200 can be run. In some embodiments, three electrophoresis cassettes comprising samples and one buffer dam can be run.

A variety of electrophoresis buffers are known in the art. Skilled artisans can select electrophoresis buffers based on the composition of gels in an electrophoresis cassette, the nature of the biomolecule to be electrophoresed and other factors. In some embodiments, both the cathode buffer chamber and the anode buffer chamber generally will have the same electrophoresis buffer. However, in specialized applications such as specialized gel chemistries as well as applications such as Isoelectric Focusing (IEF) gels and NativePAGE gels, cathode dam/chamber buffer and anode dam/chamber buffers are different in order to facilitate the pH focusing for IEF and the separation by molecular weight for NativePAGE.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literatures and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art in light of the present teachings.

Figures and drawings provided in this specification will be used to describe exemplary embodiments. One of skill in the art will note that the drawings and examples are merely for illustration of ideas and concepts and are not intended to limit the scope of the present teachings in any way. All parts are not labeled in each figure and, unless noted, similar parts have similar numbering which may not be described in each figure or every part of the description.

### EXAMPLES

Aspects of the present teachings can be further understood in light of the following examples, which should not be construed as limiting the scope of the present teachings in any way.

### Example 1

### Electrophoresis Using Electrophoresis Clamp Assemblies

Electrophoresis was conducted using exemplary electrophoresis clamp assemblies and systems of the present disclosure and successful separation of biomolecules was demonstrated for a variety of gel types such as those listed below:
4-12% NuPAGE Bis-Tris, 10-well (Thermo Fisher Scientific Cat #NP0321BOX)
4-12% Bolt Bis-Tris Plus, 10-well (Thermo Fisher Scientific Cat #NW04120BOX)
4-20% Novex Tris-Glycine Plus, 10-well (Thermo Fisher Scientific Cat #XP04200BOX)
3-8% NuPAGE Tris-Acetate, 10-well (Thermo Fisher Scientific Cat #EA0375BOX)
10-20% Novex Tricine, 10-well (Thermo Fisher Scientific Cat #EC6625BOX)

Samples were loaded in 10 lanes as described in Table 1, electrophoresed, and stained according to the Simple Blue Staining Protocol outlined below.

TABLE 1

| Lane Number | Gels 10 well | μL/well |
|---|---|---|
| Lane 1 | PageRuler Unstained | 5 μL |
| Lane 2 | HEK293, 35 μg | 5 μL |
| Lane 3 | HEK293, 30 μg | 5 μL |
| Lane 4 | HEK293, 25 μg | 5 μL |
| Lane 5 | HEK293, 20 μg | 5 μL |
| Lane 6 | HEK293, 15 μg | 5 μL |
| Lane 7 | HEK293, 10 μg | 5 μL |
| Lane 8 | HEK293, 5 μg | 5 μL |
| Lane 9 | HEK293, 1 μg | 5 μL |
| Lane 10 | PageRuler Unstained | 5 μL |

Note:
PageRuler ™ Unstained Broad Range Protein Ladder (Thermo Fisher Scientific Cat #26630) is a mixture of 11 proteins (5 to 250 kDa) for use as size standards in protein electrophoresis and HEK293 is an immortalized human embryonic kidney cell lysate lysed in RIPA lysis buffer (Thermo Fisher Scientific Cat #89901) and prepared using the recommended SDS-PAGE sample preparation for each gel chemistry. Electrophoresis gels were visualized using SimplyBlue ™ SafeStain (Thermo Fisher Scientific Cat # LC6065) staining using the protocol outlined below.

### SimplyBlue Sate Stain Protocol:

1. After electrophoresis, place the gel in 100 mL of ultrapure water in a loosely covered container, then microwave at high power (950 to 1,100 watts) for 1 minute or until the solution almost boils.
2. Agitate the gel on an orbital shaker for 1 minute (2 minutes), then discard the water.
3. Repeat step 1 and step 2 of this procedure 2 additional times.
4. After the last wash, add 20 mL (30 mL) of SimplyBlue™ SafeStain, then microwave at high power for 45 seconds to 1 minute (1.5 minutes) or until the solution almost boils.
5. Agitate the gel on an orbital shaker for 5 minutes (10 minutes). (Detection limit: 20 ng BSA.)
6. Wash the gel in 100 mL of ultrapure water for 10 minutes on a shaker. (Detection limit: 10 ng BSA.)
7. Add 20 mL of 20% NaCl, then incubate for a minimum of 5 minutes. (Detection limit: 5 ng BSA.)

Identical electrophoresis was also run on the same gels as above, with identical staining conditions, in a Mini-Gel Tank (Thermo Fisher Scientific Cat #A25977).

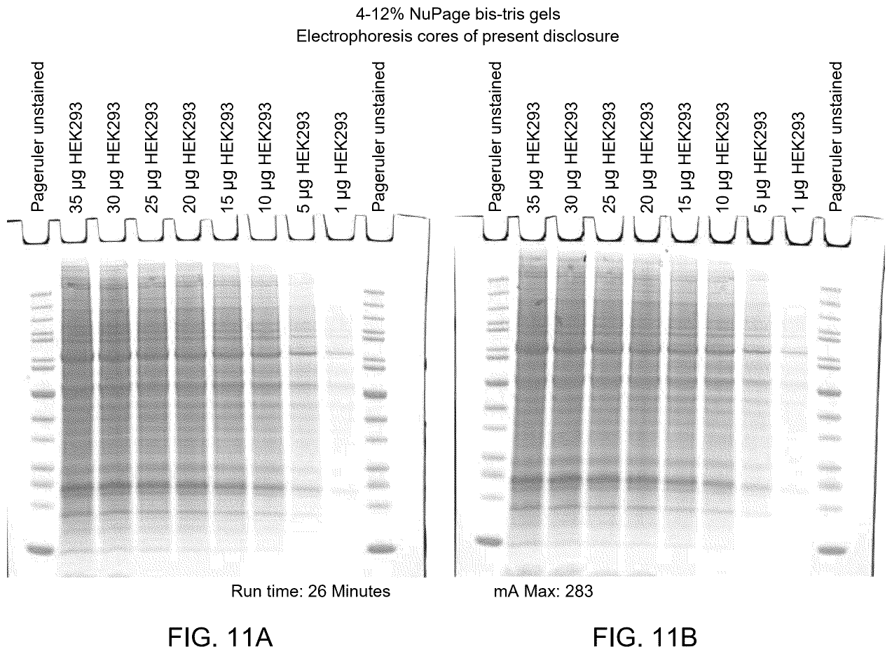

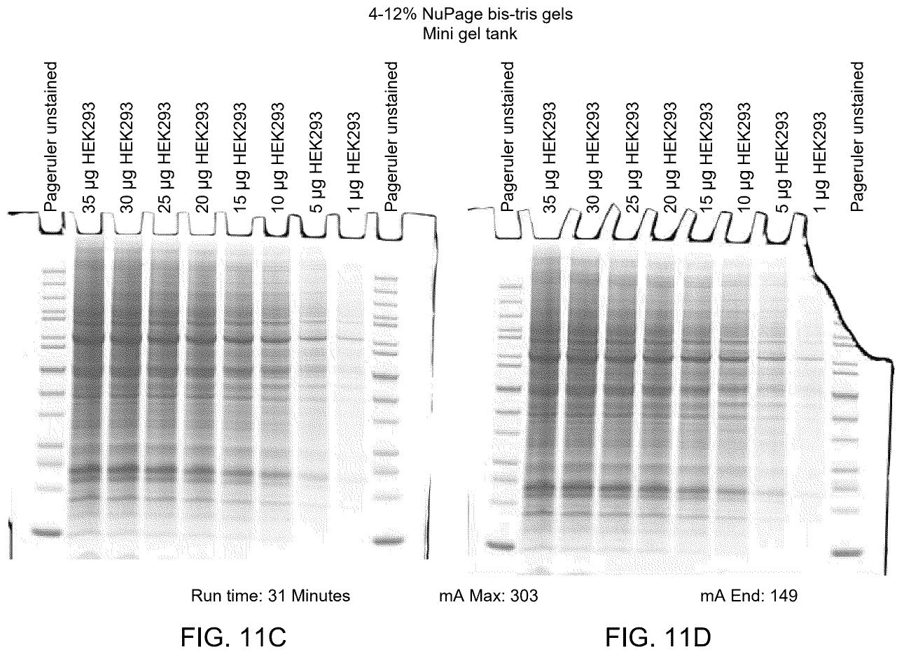

Results showing successful electrophoresis in 4-12% NuPAGE Bis-Tris gels, of the above samples in an electrophoresis core of the present disclosure compared with an electrophoresis run in a Mini-Gel Tank are shown in FIGS. 11A, 11B, 11C, and 11D. FIG. 11A and FIG. 11B, depict successful electrophoresis in 4-12% NuPAGE Bis-Tris gels in an electrophoresis core of the present disclosure, while FIG. 11C and FIG. 11D show comparative results of electrophoresis of identical samples in 4-12% NuPAGE Bis-Tris gels when the electrophoresis was run in a Mini-Gel Tank.

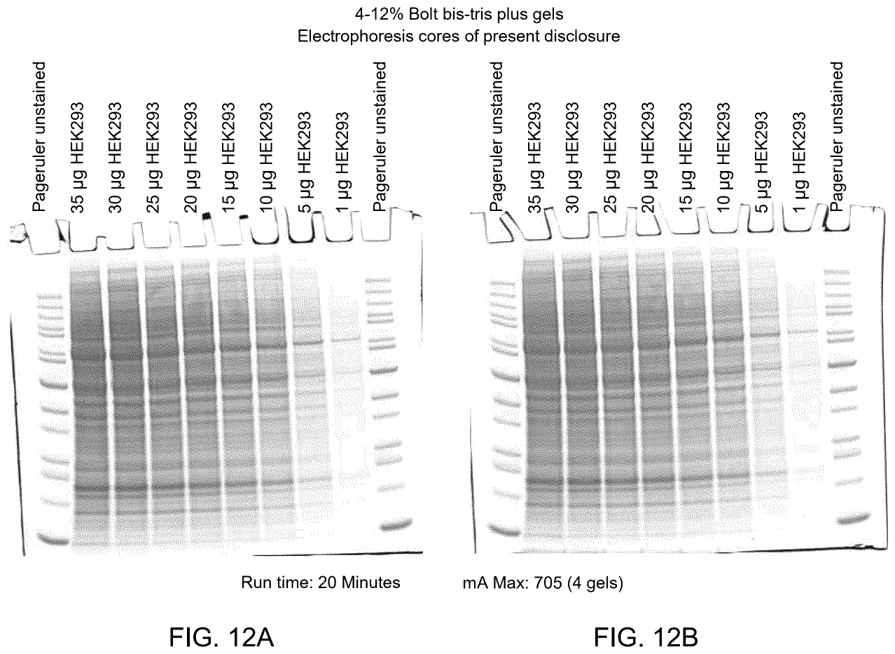

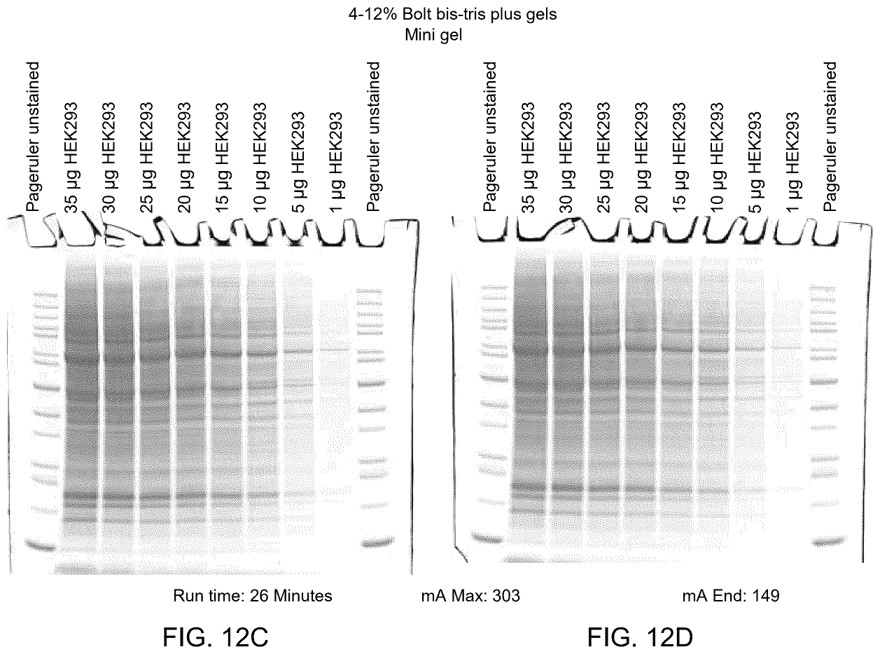

Results showing successful electrophoresis in 4-12% Bolt Bis-Tris Plus gels, of the above samples in an electrophoresis core of the present disclosure compared with an electrophoresis run in a Mini-Gel Tank are shown in FIGS. 12A, 12B, 12C, and 12D. FIG. 12A and FIG. 12B, depict successful electrophoresis in 4-12% Bolt Bis-Tris Plus gels in an electrophoresis core of the present disclosure, while FIG. 12C and FIG. 12D show comparative results of electrophoresis of identical samples in 4-12% Bolt Bis-Tris gels when the electrophoresis was run in a Mini-Gel Tank.

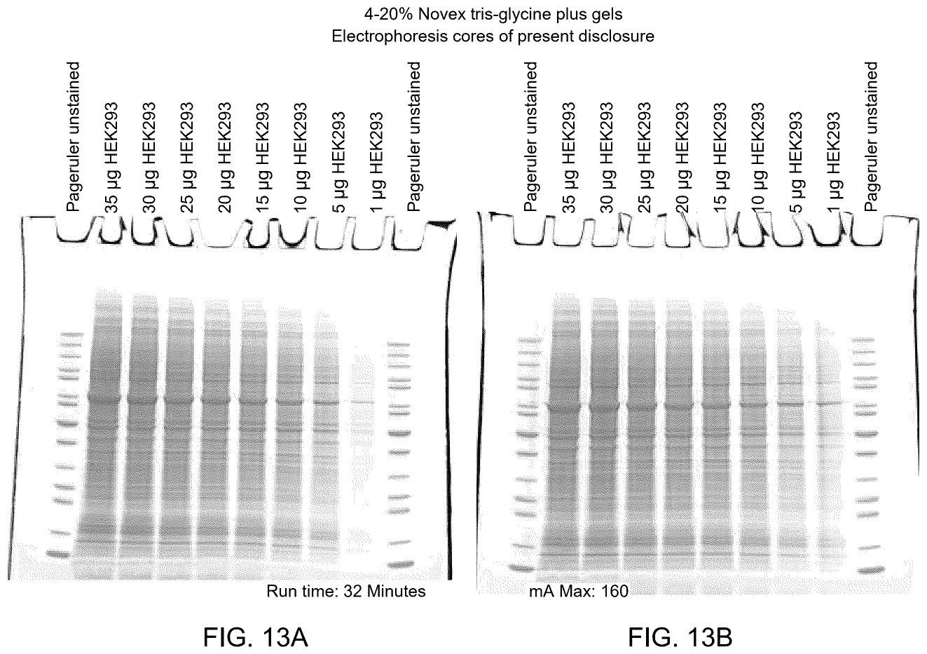

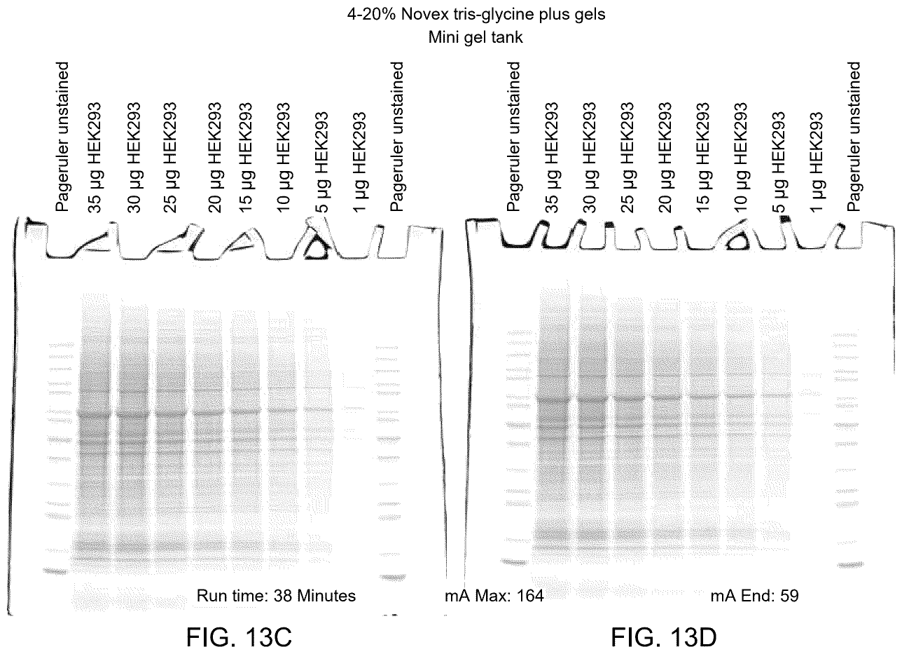

Results showing successful electrophoresis in 4-20% Novex Tris-Glycine Plus gels, of the above samples in an electrophoresis core of the present disclosure compared with an electrophoresis run in a Mini-Gel Tank are shown in FIGS. 13A, 13B, 13C, and 13D. FIG. 13A and FIG. 13B, depict successful electrophoresis in 4-20% Novex Tris-Glycine Plus gels in an electrophoresis core of the present disclosure, while FIG. 13C and FIG. 13D show comparative results of electrophoresis of identical samples in 4-20% Novex Tris-Glycine Plus gels when the electrophoresis was run in a Mini-Gel Tank.

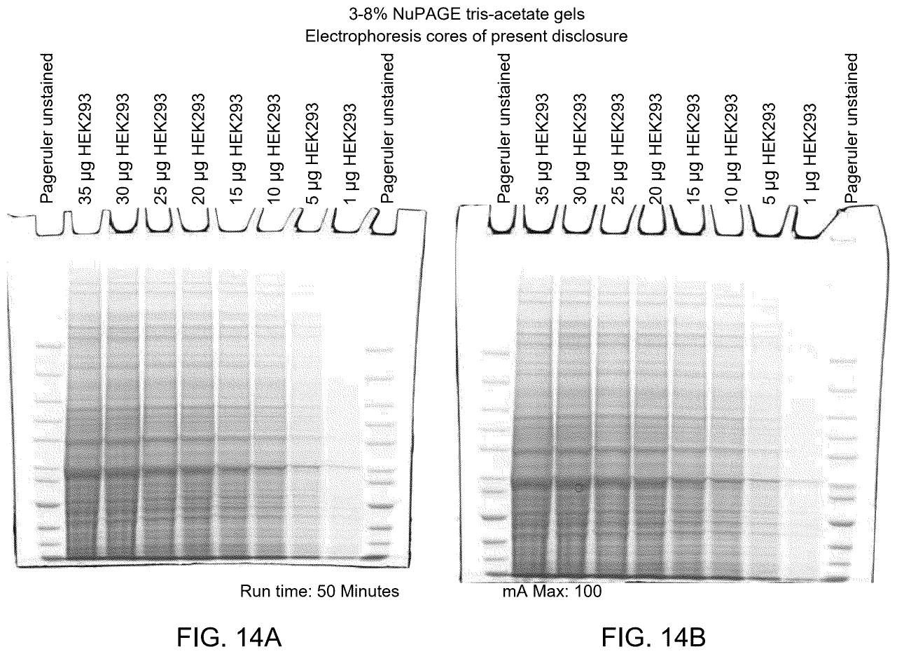

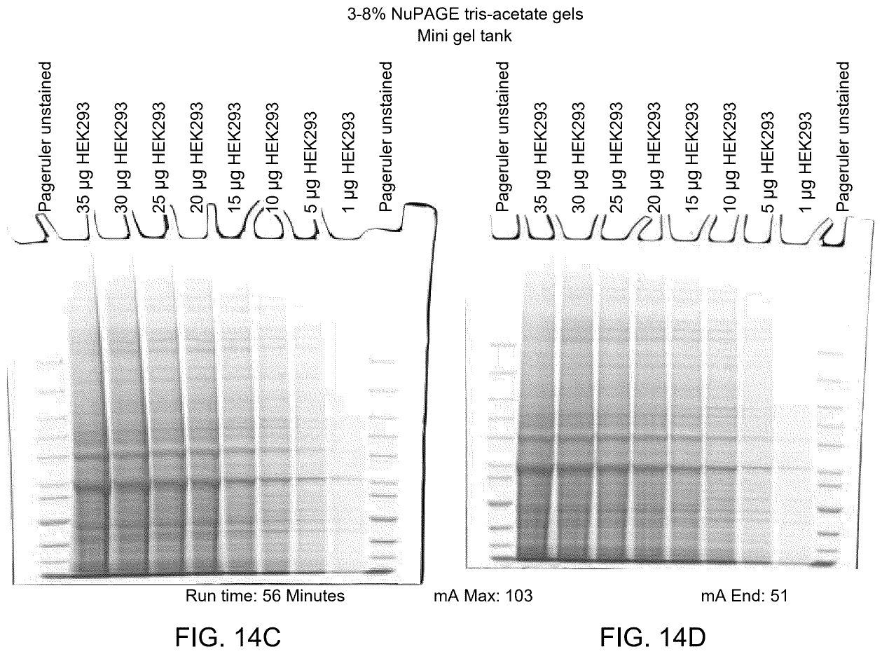

Results showing successful electrophoresis in 3-8% NuPAGE Tris-Acetate gels, of the above samples in an electrophoresis core of the present disclosure compared with an electrophoresis run in a Mini-Gel Tank are shown in FIGS. 14A, 14B, 14C, and 14D. FIG. 14A and FIG. 14B, depict successful electrophoresis in 3-8% NuPAGE Tris-Acetate gels in an electrophoresis core of the present disclosure, while FIG. 14C and FIG. 14D show comparative results of electrophoresis of identical samples in 3-8% NuPAGE Tris-Acetate gels when the electrophoresis was run in a Mini-Gel Tank.

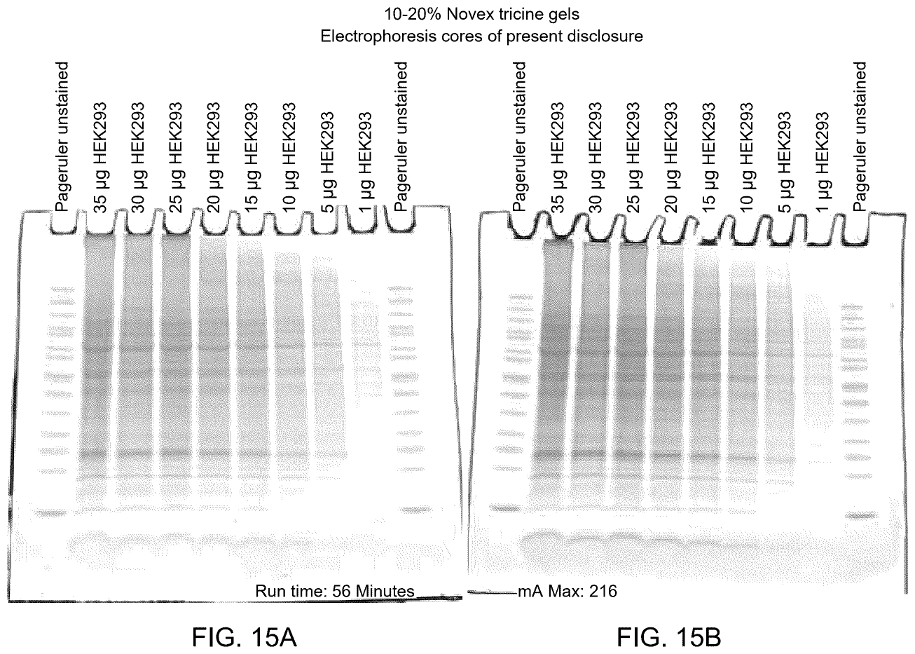

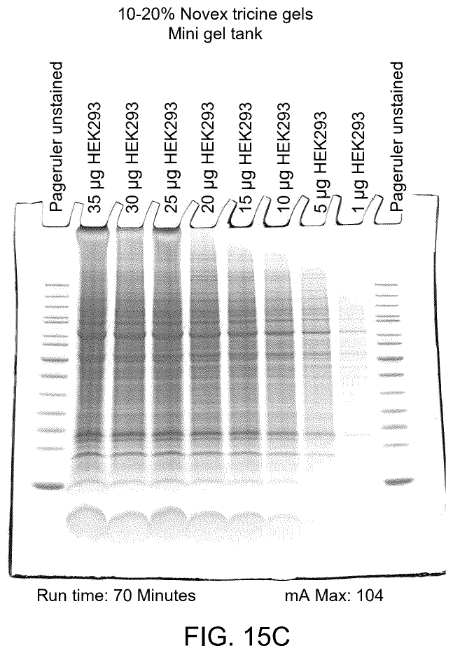

Results showing successful electrophoresis in 10-20% Novex Tricine gels, of the above samples in an electrophoresis core of the present disclosure compared with an electrophoresis run in a Mini-Gel Tank are shown in FIGS. 15A, 15B, and 15C. FIG. 15A and FIG. 15B, depict successful electrophoresis in 10-20% Novex Tricine gels in an electrophoresis core of the present disclosure, while FIG. 15C shows comparative results of electrophoresis of identical samples in 10-20% Novex Tricine gels when the electrophoresis was run in a Mini-Gel Tank.

As can be seen in all the results and FIGS. 11A-15C noted in this example, run times for the electrophoresis clamp assemblies are significantly reduced as compared to the existing Mini Gel Tank.

All publications, patents, and patent applications mentioned in this specification are herein incorporated by reference to the same extent as if each individual publication, patent, or patent application was specifically and individually indicated to be incorporated by reference.

While embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. An electrophoresis clamp assembly comprising:
a body;
an electrode assembly comprising an anode and a cathode; and
a pair of clamps,
wherein the body comprises a bottom part, that is contiguous with two upright parts on the left and right sides, and a horizontal part that spans the center and is contiguous with portions of the two upright parts.

2. The electrophoresis clamp assembly of claim 1,
wherein the bottom ends of the clamps are flared.

3. The electrophoresis clamp assembly of claim 1,
wherein the clamps have a pair of grips on the top.

4. The electrophoresis clamp assembly of claim 1,
wherein the cathode comprises a bar shaped metal and wherein the anode is a platinum wire.

5. The electrophoresis clamp of claim 4, wherein the cathode further comprises a protrusion.

6. (canceled)

7. The electrophoresis clamp assembly of claim 1, wherein the bottom area is a flat surface.

8. The electrophoresis clamp assembly of claim 1, wherein the clamps are wing clamps.

9. The electrophoresis clamp assembly of claim 1, wherein the clamps are adapted to reversibly engage with portions of the body.

10. (canceled)

11. The electrophoresis clamp assembly of claim 9, wherein the clamps are adapted to enclose one or more electrophoresis cassettes when the clamps are engaged with portions of the body.

12. The electrophoresis clamp assembly of claim 1, further comprising at least a pair of base tabs that can accommodate one or more electrophoresis cassettes.

13. The electrophoresis clamp assembly of claim 1, adapted to clamp from one to four electrophoresis cassettes.

14. The electrophoresis clamp assembly of claim 12, wherein at least one chamber is formed between the walls of the one or more electrophoresis cassettes and one or more body portions of the clamp assembly.

14.-16. (canceled)

17. The electrophoresis clamp assembly of claim 1, wherein the cathode is connected to at least one of the upright parts of the body and spans across the horizontal center part of the body.

18.-19. (canceled)

20. An electrophoresis system comprising:
at least one electrophoresis clamp assembly of claim 1; and
an electrophoresis tank; and
at least two electrophoresis cassettes.

21. The electrophoresis system of claim 20, wherein up to four electrophoresis cassettes can be run.

22. The electrophoresis system of claim 20, wherein one of the electrophoresis cassettes is a buffer dam.

23. A method comprising:
clamping at least two electrophoresis cassettes to an electrophoresis clamp assembly of claim 1;
placing the clamped electrophoresis clamp assembly comprising the electrophoresis cassettes into an electrophoresis tank;
filling the electrophoresis tank with at least one electrophoresis buffer;
loading at least one of the two electrophoresis cassettes with a sample comprising a biomolecule and optionally a loading dye;
placing an electrophoresis tank lid onto the electrophoresis tank in such a way that the anode and cathode electrodes are connected in the right polarity to an external power supply via metal connectors in the tank lid,
switching on the power supply and running the electrophoresis till the sample biomolecules have been resolved.

24.-30. (canceled)

31. An electrophoresis clamp assembly comprising:
a body;
an electrode assembly comprising an anode and a cathode; and
a pair of clamps, wherein the bottom ends of the clamps are flared.

32. An electrophoresis clamp assembly comprising:
a body;
an electrode assembly comprising an anode and a cathode; and
a pair of clamps, wherein the clamps have a pair of grips on the top.
